(19) United States
(12) Patent Application Publication
(10) Pub. No.: US 2011/0014296 A1
(43) Pub. Date: Jan. 20, 2011

(54) Drug Delivery Nanodevice, its Preparation Method and Uses Thereof

(75) Inventors: San-Yuan Chen, Hsinchu City (TW); Shang-Hsiu Hu, Pingjhen City (TW); Dean-Mo Liu, Jhubei City (TW); Kun-Ting Kuo, Dashu Township (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: NATIONAL CHIAO TUNG UNIVERSITY, Hsinchu (TW)

(21) Appl. No.: 12/504,926

(22) Filed: Jul. 17, 2009

(51) Int. Cl. A61K 9/14; A61K 38/02

(52) U.S. Cl. 424/490; 424/497; 424/9.3; 424/9.4; 977/774; 514/6.9; 514/9.6

(57) ABSTRACT

Nanodevice and method for in vivo monitoring and release of drugs are provided. The disclosed nanodevice is characterized in having a drug-loaded nanosphere that is capable of releasing the encapsulated drugs upon magnetically stimulation. The nanodevice may also be used as a contrast agent for in vivo imaging and monitoring the concentration and distribution of the released drugs and/or active compounds injected separately into a target site of a subject.

### BACKGROUND

1. Technical Field

The disclosure relates to a drug delivery nanodevice, its preparation method and uses thereof.

2. Description of Related Art

Medication can be delivered to a patient through a variety of methods, including oral ingestion, inhalation, transdermal diffusion, subcutaneous and intramuscular injection, parenteral administration and implants. Oral drug delivery remains the most preferred way of administration of a medication. However, many current drug delivery products such as oral capsules and tablets possess drawbacks such as limited effectiveness on controlled drug delivery that results in too rapid and incomplete absorption of the drug, irritation of the gastrointestinal tract and other side effects. Further, they may not provide localized therapy, and/or real time monitoring of the distribution of the released drug. Thus, there is an increasing need of an improved drug delivery system and/or device to deliver medication to patients more efficiently and with fewer side effects, while at the same time allowing in vivo tracking of the released drugs in the target site of a patient.

A variety of imaging techniques have been developed to trace metallic nanoparticles such as gold nanoparticles in vivo. These imaging techniques are used to produce images that reflect the different densities of structures and tissue in the body of a subject. The most commonly adopted imaging techniques include X-ray imaging, computed tomography (CT) and magnetic resonance imaging (MRI).

This invention designs, manufactures, and employs a novel nanodevice as a carrier for drug delivery that can be actively and remotely released by proper stimulation at a desired body portion of a subject, while tracing the nanodevice at the same time by suitable conventional imaging techniques as described above.

### SUMMARY

This disclosure relates to a drug delivery nanodevice, its manufacturing method and uses thereof. The drug delivery nanodevice includes a nanosphere, which is characterized in having a core-shell structure. The core phase may have drugs or biologically active substances encapsulated within, and the shell surface is made of a magnetic substance and has quantum dot(s) deposited thereon. This unique structure of the nanodevice of this disclosure allows the encapsulated drugs or biologically active substances to be released in a controlled manner by magnetically stimulating the magnetic shell to trigger shell deformation and thereby releasing drugs in accordance with the strength and/or duration of the applied magnetic field, whilst the quantum dots underwent optical variation. Furthermore, the nanodevice may also be traced in vivo by any suitable imaging technique such as X-ray imaging, computed tomography (CT) or magnetic resonance imaging (MRI), with or without further addition of a contrast agent.

In a first aspect of this disclosure, there provides a method of manufacturing a drug delivery nanodevice. The method includes steps of: (a) providing a first solution by dispersing a nanosphere in a first solvent containing therein a zinc salt; (b) providing a second solution by mixing at least two quantum dot precursors in a second solvent; and (c) mixing the first solution with the second solution and thereby forming a quantum dot on the surface of the nanosphere. The first solvent is a mixture formed by any two solvents selected from the group consisting of trioctylphosphine (TOP), tetrahydrofuran (THF), C₆₋₁₈ alkylene, and dimethylsuloxide (DMSO); and the second solvent is an alkylimine such as oleylamine or hexadecylamine. The quantum dot precursors are at least two materials selected from the group consisting of cuprous chloride (I), indium trichloride (III), indium iodide (III), sulfur powder, zinc stearate, cadmium chloride (II), and Te powder. The quantum dot is paramagnetic and is any of CuInZn, CuInS₂, CdS, ZnS or CdTe.

According to one embodiment, the nanosphere is formed by steps of: (a) providing a suspension by mixing a polymeric material, an inorganic material or a combination thereof with a drug in a polar solvent and thereby forming a drug-containing nanoparticle; and (b) adding at least two metal oxide precursors to the suspension; wherein the at least two metal oxide precursors self-assemble into a metal oxide outer shell around the drug-containing nanoparticle. In one embodiment, the polymeric material is selected from the group consisting of polyvinylpyrrolidone (PVP), polyethylene (PE), polyamide, polyester, polyanhydride, polyether, poly acetal, polysaccharide and phospholipid; and the inorganic material is selected from a group consisting of titania, silica and a compound material made of calcium and phosphate. The metal oxide outer shell is a single crystal shell, poly-crystal shell or an amorphous shell that comprises any of Fe₂O₃, Fe₃O₄, CoFe₂O₄, MnFe₂O₄ or Gd₂O₃.

In a second aspect of this disclosure, there provides a nanodevice. The nanodevice includes a nanosphere and a quantum dot. The nanosphere comprises a core made of a polymeric material, an inorganic material or a combination thereof; and an outer shell made of a metal oxid. The quantum dot is deposited on the surface of the outer shell, and is selected from the group consisting of CuInZn, CuInS₂, CdS, ZnS and CdTe. The drug is any of an anti-epileptic agent, an anti-tumor agent, an anti-bacterial agent, an anti-viral agent, an anti-proliferative agent, an anti-inflammatory agent, an anti-diabetic agent, or a hormone.

In a third aspect of this disclosure, there provides a method of in vivo imaging and magnetically induced drug release from the nanodevice prepared in accordance with the method described above. The method includes steps of: (a) administering a sufficient amount of the nanodevice of this invention to a body portion of the subject; and (b) magnetically stimulating the body portion of the subject with a high frequency magnetic field from about 0.05 kA/m to 2.5 kA/m for a period of about 10 to 180 sec, such that the nanodevice releases the encapsulated drug into the magnetically stimulated body portion of the subject. The method may further comprise a step of: tracing the body portion of the subject by an imaging method selected from the group consisting of X-ray imaging, computed tomography (CT), and magnetic resonance imaging (MRI) without further addition of a contrast agent. The body portion may be the brain area of a human.

These and other features, aspects, and advantages of the present invention will become better understood with reference to the following description and appended claims.

It is to be understood that both the foregoing general description and the following detailed description directed to the uses and application of such nanodevice are not strictly limited to the ranges described in those examples, and are intended to provide further explanation of the invention as claimed.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The accompanying drawings are included to provide a further understanding of the invention, and are incorporated in and constitute a part of this specification. The drawings illustrate embodiments of the invention and, together with the description, serve to explain the principles of the invention. In the drawings,

FIGS. 1(a) and 1(b) are schematic diagrams of a drug delivery nanodevice and a nonosphere, respectively in accordance with one example of this disclosure;

FIG. 2(a) is a schematic view of the process for manufacturing a nanodevice comprising a single crystal Fe₃O₄ shell self-assemblies around a polymeric core in accordance with one example of this disclosure;

FIGS. 2(b) and 2(c) are transmission electron microscopy (TEM) photograph and high resolution transmission electron microscopy (HRTEM) photograph of nanospheres prepared in accordance with one example of this disclosure;

FIG. 2(d) is the HRTEM photograph of the nanodevice prepared in accordance with one example of this disclosure, in which solid particle of ZCIS QDs were deposited on the ring-like shell of the nanosphere, and the suspension was able to produce fluorescene under UV light (inset picture);

FIG. 3 illustrates respective field-dependent magnetization curve of the nanodevice and the nanosphere prepared in accordance with one example of this disclosure;

FIG. 4(a) gives the emission spectra of the model drug-loaded nanodevices (30 mg/10 ml water) under HFMF treatment over a period of 0 s to 100 s;

FIG. 4(b) gives the relationship between the model drug intensity and the quantum dot intensity originated from both the FITC dye and ZCIS emitting spectra under various strengths of magnetic field;

FIG. 5 are fluorescent photographs taken from Hela cells treated with FITC-loaded nanodevices after 12 hours in accordance with one example of this disclosure, in which Gₛᵤₘ/Bₛᵤₘ represents the ratio of green channel intensity to the blue channel intensity, and is an indicative of the relative concentration of the model drug in each cell, and Rₛᵤₘ/Bₛᵤₘ is defined as the relative intensity of the nanodevice in each cell;

FIG. 6 illustrates the ratio of Gₛᵤₘ/Bₛᵤₘ and Rₛᵤₘ/Bₛᵤₘ versus the urations of magnetic stimulus in cells of FIG. 4;

FIG. 7 illustrates the uptake of the nanodevices into cells in accordance with one example of this disclosure; the photograph was taken 2 hours after treatment;

FIG. 8 illustrates the cytotoxicity of the nanospheres and nanodevices measured in cells that were treated with the nanospheres and nanodevices respectively for 12, 24 and 48 hours in accordance with one example of this disclosure;

FIG. 9 illustrates the cell viability of cancerous cells treated with CPT-loaded nanospheres and nanodevices for 12, 24 and 48 hours respectively in accordance with one example of this disclosure;

FIG. 10 illustrates the uptake of the CPT-laoded nanodevices in cells that were pretreated with the CPT-loaded nanodevices and magnetically stimulated with HFME for various durations in accordance with one example of this disclosure;

FIG. 11 illustrates spontaneous SWDs recorded in animals treated with (a) slaine, (b) ethosuximide (ESM) (28 mg/Kg, i.p.), (c) ESM-conatining nanoparticles (ESM-SAIO) (48 mg/Kg, i.p.) and (d) ESM-containing nanodevice (ESM-device) (40 mg/Kg, i.p.) in accordance with one example of this disclosure;

FIG. 12 illustrates the comparision of SWD number and total SWD duration measured in animals treated with saline, ESM (0.5 ml, 28 mg/Kg, i.p.), ESM-containing nanoparticles (ESM-SAIO) (40 mg/Kg, i.p.) and ESM-containing nanodevices (ESM-device) (40 mg/Kg, i.p.), respectively in accordance with one example of this disclosure;

FIG. 13 are T₂-weighted images of a rat brain area taken before (upper row) and after (lower row) injection of the nanodevices in accordance with one example of this disclosure;

FIG. 14 are the dynamic contrast-enhanced MRI of the rat brain area of FIG. 12, in which (A) is a T₂-weighted image after 30-min injection of the nanodevice; (B) and (C) are the signal profiles in the right and left brain hemisphere, respectively, and the red arrow indicated the time stamp of injection of said nanodevice; and

FIG. 15 are MRI images of the rat brain area taken after amphetamine stimulation and its activated time course, in which the event related activations were labeled by hot and cold colors for correlation coefficients that were +0.5 and under −0.5, respectively.

### DETAIL DESCRIPTION OF THE DISCLOSURE

Reference will now be made in detail to the present embodiments of the invention, examples of which are illustrated in the accompanying drawings.

Described below is a nanodevice for in vivo imaging and/or magnetically induced drug release, its preparation method and uses thereof. The novel nanodevice may be actively and remotely controlled to release encapsulated drug in a desired body portion of a subject, such as the brain area or any other organ of a human; and the nanodevice may be traced in vivo by a suitable imaging technique with or without further addition of a conventional contrast agent such as barium sulfate, iodine based contrast agent, indium, gadolinium, iron oxide, and/or manganese chelates iron oxide.

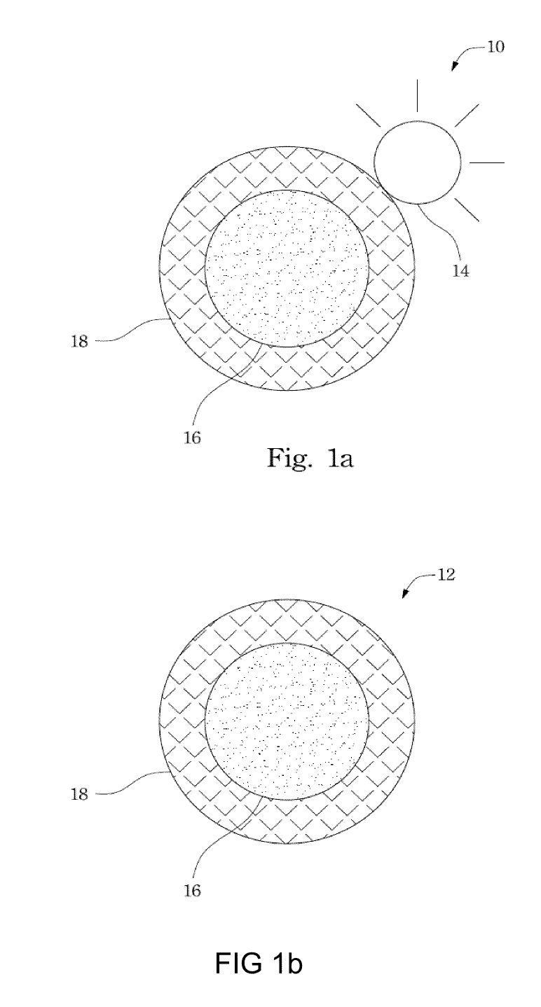

Referring to FIG. 1(a), which is a schematic diagram of a drug delivery nanodevice 10 of this disclosure. The nanodevice 10 is composed of a nanosphere 12 as illustrated in FIG. 1(b), and a quantum dot 14. In this example, only one quantum dot 14 is shown, however, it should be noted that multiple quantum dots 14 may be present on the nanosphere 12, if necessary. The nanosphere 12 is characterized in having a core/shell structure. The core 16 may be made of a polymeric material, an inorganic material or a combination thereof; and the outer shell 18 is made of a metal oxide. The quantum dot 14 is deposited on the surface of the outer shell 18. The nanodevice 16 is configured to encapsulate a drug in the polymeric, inorganic or polymeric/inorganic core of the nanosphere 12. The encapsulated drug may be released from the core 16 in a controlled manner by magnetically stimulating the outer shell 18 with a magnetic field (MF) to generate shell deformation and/or to collapse of the metal oxide shell 18 and thereby releasing the encapsulated drug 20.

### Preparation of Nanospheres

The nanosphere described above may be prepared by a method described previously (Hu et al., “Core/Single-Crystal-Shell Nanospheres for Controlled Drug Release via a Magnetically Triggered Rupturing Mechanism” Adv. Mater., 2008, 20, 2690-2695); its entire disclosure is incorporated herein by reference. In one embodiment, the nanosphere is formed by steps of: providing a suspension by dissolving about 1-10% (wt %) of a polymeric material, an inorganic material or a combination thereof in a polar solvent such as water or a C₁₋₆ alcohol and thereby forming a polymeric, inorganic or polymeric/inorganic nanoparticle; and adding at least two metal oxide precursors to the suspension; wherein the at least two metal oxide precursors self-assembled into a metal oxide outer shell around the nanoparticle.

Suitable polymeric material that may be used for forming the core phase of the nanosphere includes, but is not limited to, polyvinylpyrrolidone (PVP), polyethylene (PE), polyamide, polyester, polyanhydride, polyether, poly acetal, polysaccharide and phospholipid. The polysaccharide is any of starch, cellulose, pectin, chitin or chitosan; and the phospholipid is any of phosphatidylcholin (PC), phosphatidylserine (PS), phosphatidylethanolamine, phosphatidylglyerol or phosphatidyllechithin. In one example, the polymeric material is PVP. Suitable inorganic material includes, but is not limited to, titania, silica and a compound material made of calcium and phosphate. In one example, the inorganic material is silica ; and in another example, the inorganic material is titania.

Suitable metal oxide precursors for assembling around the polymeric or inorganic nanoparticle and thereby forming a metal oxide outer shell include, but are not limited to, ferrous chloride (II), ferric chloride (III), cobalt chloride (II), ferrous nitrate (II), ferric acetate (III), cobalt acetate (II), gadolinium chloride (III), and manganous acetate (II). The metal oxide outer shell may be a single crystal shell, poly-crystal shell or an amorphous shell that comprises any of Fe₂O₃, Fe₃O₄, CoFe₂O₄, MnFe₂O₄ or Gd₂O₃. In one example, the metal oxide outer shell is a single-crystal iron oxide shell formed by steps of: mixing at least two metal oxide precursors comprise ferrous chloride (II) and ferric chloride (III) in a molar ratio of about 2:1 to 5:1 in a polar solvent such as water or a C₁₋₆ alcohol; adjusting pH to a range between 7 and 12; and allowing the thereformed iron oxide to self-assemble around the nanoparticle. The C₁₋₆ alcohol may be selected from the group consisting of methanol, ethanol, propanol, isopropanol, butanol, isobutanol, sec-butanol, pentanol, isopentanol, hexanol and the like. Other metal oxide shell may be prepared by the steps described above using suitable metal oxide precursors. For example, CoCl₂ and FeCl₃ may be used for the preparation of CoFe₂O₄ outer shell; MnCl₂ and FeCl₃ may be used for the preparation of MnFe₂O₄ outer shell; and Gd acetate or Gd(OH)₂ may be used for the preparation of Gd₂O₃ outer shell.

If a drug-loaded nanosphere is desired, then suitable amounts of a drug or a biologically active substance may be further added to the above described suspension and mixed thoroughly, so that the drug is encapsulated within the structure of the polymeric material or inorganic material; and a metal oxide outer shell may subsequently assemble around the drug-containing nanoparticle and thereby forming the drug-loaded nanosphere. The amount of the drug that may be encapsulated with the core phase of the nanoparticle is usually determined empirically, depends on particular type of drug intended to be encapsulated therein. The term “drug” or “biologically active substance” may be used interchangeably herein, and refers to a compound or composition useful for the treatment and/or prevention of conditions in a variety of therapeutic areas and can be administered to a living organism, especially animals such as mammals, particularly humans. The drug useful herein includes, but is not limited to, nucleic acids such as DNA or small interference RNA (siRNA); peptides; proteins such as bovine serum albumin, glycoproteins or collagens; antibiotics; antioxidants such as vitamin E or vitamin C (i.e., ascorbic acid); immunogenic preparations such as a vaccine preparation; an anti-epileptic agent, such as acetazolamide, carbamazepine, clobazam, clonazepam, diazepam, ethosuximide, ethotoin, felbamate, fosphenytoin, gabapentin, lamotrigine, levetiracetam, mephenytoin, metharbital, methsuximide, methazolamide, oxcarbazepine, phenobarbital, phenytoin, phensuximide, pregabalin, primidone, sodium valproate, stiripentol, tiagabine, topiramate, trimethadione, valproic acid, vigabatrin or zonisamide; an anti-tumor agent such as taxol, camptothecin (CPT), topotecan (TPT) or irinotecan (CPT-11); an anti-bacterial agent such as zinc oxide or quaternary ammonium compounds; an anti-viral agent such as acyclovir, ribavirin, zanamivir, oseltamivir, zidovudine or lamivudine; an anti-proliferative agent such as actinomycin, doxorubicin, daunorubicin, valrubicine, idarubicin, epirubicin, bleomycin, plicamycin or mitomycin; an anti-inflammatory agent such as orticosteroids, ibuprofen, methotrexate, aspirin, salicyclic acid, diphenyhydramine, naproxen, phenylbutazone, indomethacin or ketoprofen; an anti-diabetic agent, which includes sulfonylureas such as tolbutamide, acetohexamide, tolazamide, chlorpropamide, glipizide, glyburide, glimepiride or gliclazide; meglitinides such as repaglinide or nateglinide; biguanides such as metformin, phenformin, or buformin; thiazolidinediones such as rosiglitazone, pioglitazone or troglitazone; alpha-glucosidase inhibitors such as miglitol or acarbose; peptide analogs such as exenatide, liraglutide, taspoglatide, vildagliptin, sitagliptin or pramlintide; and a hormone such as insulin, epidermal growth factor (EGF), and steroids such as progesterone, estrogen, corticosteroids and androgens. According to one embodiment of this disclosure, the amount of drug in the nanosphere ranges from about 0.01% to 80% (wt %), such as 0.01, 0.05, 0.1, 0.2, 0.5, 1, 2, 5, 8, 10, 12, 15, 18, 20, 22, 25, 28, 30, 32, 35, 38, 40, 42, 45, 48, 50, 52, 55, 58, 60, 62, 65, 68, 70, 72, 75, 78 or 80%. Any skilled person in the medical practice would know how to choose a proper combination of drugs for magnetic field induced drug release by use of the nanodevice of this disclosure without undue experiment. In one example, the drug is an anti-epileptic agent such as ethosuximide. In another example, the drug is an anti-tumor agent such as camptothecin (CPT).

In one embodiment, the nanosphere thus prepared has an average diameter of about 10 nm to 100 nm, such as about 10, 11, 12, 13, 14, 15, 16, 17, 18, 19, 20, 30, 40, 50, 60, 70, 80, 90 or 100 nm; and an amorphous core and a single-crystal iron oxide shell structure that suggests self-organization of the iron oxide precursor upon nucleation and growth around the PVP core.

### Preparation of Nanodevices

In one aspect, the disclosure is directed to a method of manufacturing a drug delivery nanodevice. The method is characterized in having steps of:

(a) providing a first solution by dispersing a nanosphere in a first solvent containing therein a zinc salt, and the nanosphere and the zinc salt have a concentration of about 1-40 mg/ml and 0.02-0.2 mmol/ml, respectively in the first solution;

(b) providing a second solution by mixing at least two quantum dot precursors in a second solvent, wherein each of the at least two quantum dot precursors has a concentration of about 0.003-0.03 mmol/ml in the second solution; and

(c) mixing the first solution with the second solution in the presence of an inert gas at a temperature between 10° C. to 300° C. and thereby forming a quantum dot on the surface of the nanosphere.

In step (a), the first solution is made by dispersing a nanosphere in a first solvent in the presence of a zinc salt. The nanosphere may be prepared in accordance with the steps described above, and the nanosphere may or may not have encapsulated within its core phase a therapeutic agent or a drug. In one example, the nanosphere is loaded with a drug in the core phase in accordance with steps described above. In another example, the nanosphere is used as a contrast agent and therefore does not contain any drug in its core phase. The first solvent is a mixture formed by any two solvents selected from the group consisting of trioctylphosphine (TOP), tetrahydrofuran (THF), C₆₋₁₈ alkylene, and dimethylsuloxide (DMSO). In one example, the first solvent is formed by TOP and hexane. Suitable zinc salt that may be used in this disclosure includes, but is not limited to, diethyldithiocarbamic acid zinc salt. The nanospheres are dispersed in the first solution in a concentration of about 1-40 mg/ml, for example, about 1, 5, 10, 12, 15, 18, 20, 22, 25, 28, 30, 32, 35, 38 or 40 mg/ml. The zinc salt is dispersed in the first solution in a concentration of about 0.02-0.2 mmol/ml, for example, about 0.02, 0.03, 0.04, 0.05, 0.06, 0.07, 0.08, 0.09, 0.1, 0.11, 0.12, 0.13, 0.14, 0.15, 0.16, 0.17, 0.18, 0.19 or 0.2 mmol/ml.

In step (b), the second solution is made by mixing at least two quantum dot precursors in a second solvent. In one example, the second solvent is alkylimine. However, other solvent may be used as well, as long as it may provide suitable solubility to the quantum dot precursors. Suitable quantum dot precursors are choosen based on the desired quantum dot intended to be deposited on the outer shell surface of the nanosphere, suitable quantum dot in this disclosure includes, but is not limited to, CuInZn, CuInS₂, CdS, ZnS or CdTe. If the desired quantum dot is CdS, then quantum dot precursors may be cadmium chloride (II) and sulfur powder. In one example, the desired quantum dot is CuInZn, hence the selected quantum dot precursors comprise cuprous chloride (I), indium trichloride (III), indium iodide (III), and zinc stearate. In case when the desired quantum dot is CuInS₂, the quantum dot precursors may comprise cuprous chloride (I), indium trichloride (III), indium iodide (III), and sulfur powder. In another example, zinc stearate and sulfur powder are used for the deposition of ZnS quantum dot; and cadmium chloride (II) and Te powder are used for the deposition of the CdTe quantum dot. Each of the quantum dot precursors has a concentration of about 0.003-0.03 mmol/ml in the second solution, for example, a concentration of about 0.003, 0.005, 0.007, 0.009, 0.01, 0.012, 0.014, 0.016, 0.018, 0.02, 0.022, 0.024, 0.026, 0.028 and 0.03 mmol/ml.

Finally, in step (c), the first solution and the second solution are mixed in the presence of an inert gas, such as N₂, He, Ne, Ar or combinations thereof, at a temperature between 10° C. to 300° C. and thereby forming a quantum dot on the surface of the nanosphere. In one example, the temperature is set at 140° C.

The nanodevice thus prepared may be used as a drug carrier to deliver drug to any target site within a subject, such as the brain area or any organ of a human; or it may be used as a tool to image and track the pharmaco-kinetics of a drug within the target site of the subject.

In vivo Imaging and Magnetically Induced Drug Release

It is a further aspect of this disclosure to provide a method of in vivo imaging and magnetically induced drug release form the nanodevice prepared in accordance with the method described above. The method includes steps of: (a) administering a sufficient amount of the nanodevice of this invention to a body portion of the subject; and (b) magnetically stimulating the body portion of the subject with a magnetic field from about 0.05 kA/m to 2.5 kA/m for a period of about 10 to 180 sec, such that the nanodevice releases the encapsulated drug into the magnetically stimulated body portion of the subject. The method may further comprise a step of: tracing the body portion of the subject by an imaging method selected from the group consisting of electron spin resonance (ESR) imaging, X-ray imaging, computed tomography (CT), and magnetic resonance imaging (MRI) without further addition of a contrast agent.

A subject herein refers to a human and a non-human animal. Examples of a non-human animal include all vertebrates, e.g., mammals, such as primates, dogs, rodents (e.g., mouse or rat), cats, sheep, horses or pigs; and non-mammals, such as birds, amphibians, reptiles and etc. In one example, the subject is a human. The nanodevice of this disclosure may be administered to the subject systemically by ingestion or injection; regionally (e.g., to the gastrointestinal, hepatic or renal systems, for example, or to anatomic regions such as abdomen, lumbar spine, arm or leg regions) by intravenous injection or topical application; or to a specific treatment site on or in the subject's body via topical administration or injection into the target site, or by ingestion with a feeding tube. These and other possible methods of administration of the nanodevice are known to those skilled in the art and are included in this disclosure. The body portion suitable for injection is selected based on the followings, such as the choice of the active agent to be released, the subject's personal condition including sex, age, body weight, and/or current and prior medical conditions. An experienced physician may determine suitable body portion for injection without undue experiment. In one example, the body portion is an upper arm region of a human. In another example, the body portion may be the brain area of a human. Further according to one embodiment of this disclosure, the nanodevice poses no safety concerns (i.e., no toxicity) to the subject that takes in the nanodevice.

According to exemplary embodiment of the present disclosure, the drug-loaded nanodevice is introduced into a body portion of a subject, e.g., by injection, oral administration, perfusion or the like as described above. The nanodevice advantageously becomes concentrated in the organ or region of the body portion of interest, e.g., the body portion to which the encapsulated drug is to be delivered and/or for which the drug is active, such as the brain. While the nanodevice remains substantially intact, the concentration and distribution of the nanodevice in the tissue (such as the brain) may be mapped by electron spin resonance (ESR) imaging technique, which is a method of MRI in which enhancement of the magnetic resonance signals from which images may be generated is achieved by virtue of dynamic nuclear polarization that occurs usually in paramagnetic material. The mapping is generally undertaken by irradiating the body portion at the frequency of the electron transition of the contrast agent, which is the iron oxide of the nanodevice that act as a contrast agent in according to one exemplary embodiment of this disclosure. Alternatively, other contrast agent may be used together with the nanodevice of this disclosure so as to provide images with high quality. Suitable contrast agent includes, but is not limited to, barium sulfate; iodine based contrast agent such as diatrizoate, metrizoate, ioxaglate, iopamidol, iohexol, ioxilan, iopromide and iodixanol; indium; gadolinium; iron oxide; and manganese chelates.

After the initial image measurement, the encapsulated drug is then delivered to the target site by magnetically stimulation to break down and/or disintegrate the metal oxide outer shell of the nanodevice, whether in whole or in part. Specifically, the body portion is magnetically stimulated with a magnetic field for a period of time, such as from about 10 to 180 sec, so as to release the encapsulated drug and/or drug in the body portion to effect a particular therapy such as gene transcription or tumor treatment. The strength of the magnetic field (MF) ranges from about 0.05 kA/m to 2.5 kA/m, such as about 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4 or 2.5 kA/m. The duration of the applied MF may last for a period of about 10 to 180 sec, such as about 10, 20, 30, 40, 50, 60, 70, 80, 90, 100, 110, 120, 130, 140, 150, 160, 170 or 180 sec. Upon magnetically stimulation, the quantum dot deposited on the surface of the outer shell absorbs the magnetic energy and causes deformation of the metal oxide shell of the nanodevice (e.g., morphological change of the outer shell), which in term resulted in the release of the encapsulated drug from the core phase. In the most extreme condition, the metal oxide shell collapses completely after magnetically stimulation, thus the encapsulated drug is released by rupturing the nanodevice wall. Therefore, the effective amount of the drugs in the body portion may be controlled by the strength and/or duration of the MF applied. In other words, the drugs encapsulated within the nanodevice of this disclosure may be released in a controlled manner by proper adjusting the strength and/or duration of the applied MF on the body portion of the subject.

Once the encapsulated drug has been released from the core phase of the nanodevice, further measurements may be made by MRI technique, so as to plot the pharmaco-kinetics of the drug, hence, NMR signals in MRI may be used to monitor/measure the concentration and/or distribution of the released drug. For example, using NMR signals to monitor the dynamics of the drug release, or to generate a two-dimensional or three-dimensional images that show the distribution of the contrast agent (e.g., the nanodevice of the present disclosure), and assuming comparable pharmaco-kinetic properties, the associated drug.

According to another exemplary embodiment of the disclosure, an empty nanodevice (i.e., without encapsulating therein a drug) of this disclosure is used as a contrast agent to image and track pharmaco-kinetic properties of another active agent (e.g., amphetamine), which is injected afterwards after the nanodevice has been administered and concentrated in the target site of the subject.

Reference will now be made in detail to the embodiments of the invention, examples of which are illustrated in the accompanying drawings. Wherever possible, the same reference numbers are used in the drawings and the description to refer to the same or like parts.

### EXAMPLES

The following Examples are provided to illustrate certain aspects of the present invention and to aid those of skilled in the art in practicing this invention. These Examples are in no way to be considered to limit the scope of the invention in any manner.

### Example 1

### Preparation and Characterization of a Nanodevice Encapsulating Therein a Model Drug

### 1.1 Preparation of PVP-Fe₃O₄Core-Shell Nanospheres

4% (wt %) polyvinylpyrrolidone (PVP) was dissolved in distilled water and subsequently heated the PVP solution to 80° C. In this PVP solution, a green fluorescence emitting compound, fluorescein isothiocyanate (FITC, 0.01 mg), was added and mixed for 6 hrs, the PVP would automatically assemble into nanospheres with FITC being encapsulated therein and thereby forming a FITC-laoded PVP nanoparticle. Under nitrogen condition, dissolved FeCl₃.6H₂O and FeCl₂.4H₂O (wherein the molar ratio of FeCl₂/FeCl₃ is about 2:1) in water and mixed with the CPT-laoded PVP nanoparticle under vigorously stirring at 80° C. After 4 hrs, 2 ml ammonium water (NH₄OH, 33%) was slowly added to the mixture, causing precipitation of iron oxide shells on the surface of the PVP nanoparticle. The solution was then subjected to centrifugation at 6,000 rpm, the supernatant was removed and the precipitated powders was collected and washed with distilled water for at least 4 times. The PVP-Fe₃O₄ core-shell nanospheres were separated by centrifugation.

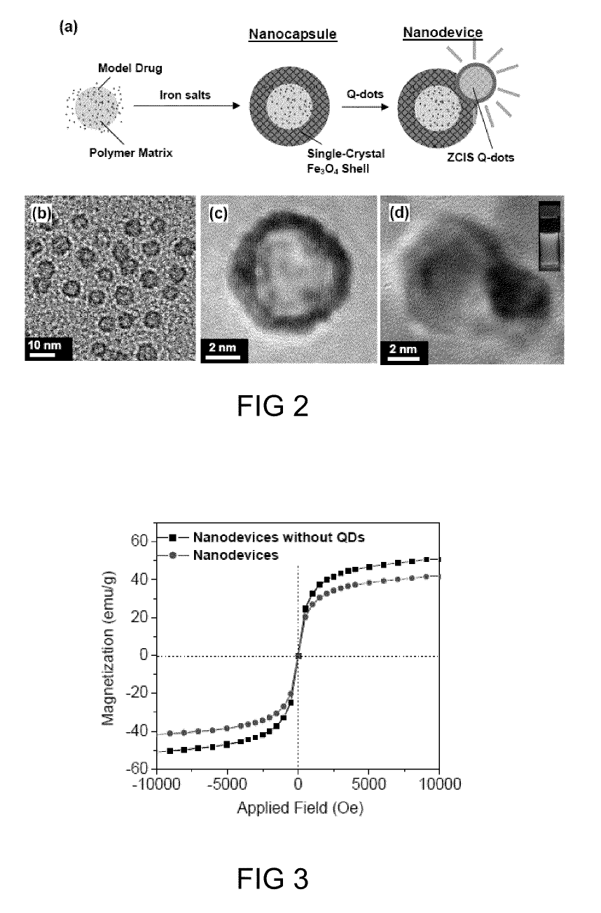

FIG. 2(a) is a schematic diagram illustrating the process of forming a drug delivery nanodevice in accordance with the steps described in this example. FIGS. 1(b) and 1(c) are transmission electron microscopy (TEM) photograph and high resolution transmission electron microscopy (HRTEM) photograph of the prepared nanospheres in this example. The results from FIGS. 2(b) and 2(c) confirm that each nanosphere has a spherical geometry ranging from 10-15 nm in diameter, and an amorphous core and a single-crystal shell structure that suggests self-organization of the iron oxide precursor upon nucleation and growth around the PVP core.

### 1.2 Deposition of Zn—Cu—In—S (ZCIS) Quantum Dots (QDs) on the Nanosphere of Example 1.1

To grow ZCIS quantum dots on the surface of the nanospheres, the nanospheres of Example 1.1 were re-dispersed in trioctylphosphine (TOP, 90%, technical grade) contained therein about 0.1-1 mmol diethyldithiocarbamic acid zinc salt, [(C₂H₅)₂NCSS]₂Zn. The solution was further diluted with octadecene (ODE, 90%, technical grade) to form Solution 1. Then, CuCl and InCl₃ were dissolved in oleylamine at 50° C. to form Solution 2. The two solutions were then mixed and heated to 140° C. in a nitrogen atmosphere for several minutes to deposit QDs on the surface of the nanospheres of Example 1.1.

FIG. 2(d) is the HRTEM photograph of the ZCIS-doped nanosphere, in which solid particle of ZCIS QDs were deposited on the ring-like shell of the nanosphere, and the suspension was able to produce fluorescene under UV light (inset picture), which suggests the nanodevice of this example can be used not only as a drug carrier, but also as a nano-probe for imaging. Energy dispersive X-ray spectrometer (EDS) analysis confirms that the ring-like region mainly consists of Fe and the solid particle mainly consists of Cu and S (data not shown).

The magnetic properties of the prepared nanodevices of Example 1.2 and the nanospheres of Example 1.1 (i.e., without formation of QDs on the surface of the nanosphere) were further investigated by superconducting quantum interference device (SQUID, MPMS-XL7) at 298K, with the magnetic field sweeping from −10000 to +10000 G. Result was provided in FIG. 3. Both the nanodevices of Example 1.2 and the nanospheres of Example 1.1 showed superparamagnetic behavior, with the nanodevices of Example 1.2 having smaller saturation magnetization (Ms) than that of the nanospheres of Example 1.1 due to dilution effect.

### 1.3 Controlled Release of the Encapsulated Model Drug from the Nanodevices of Example 1.2

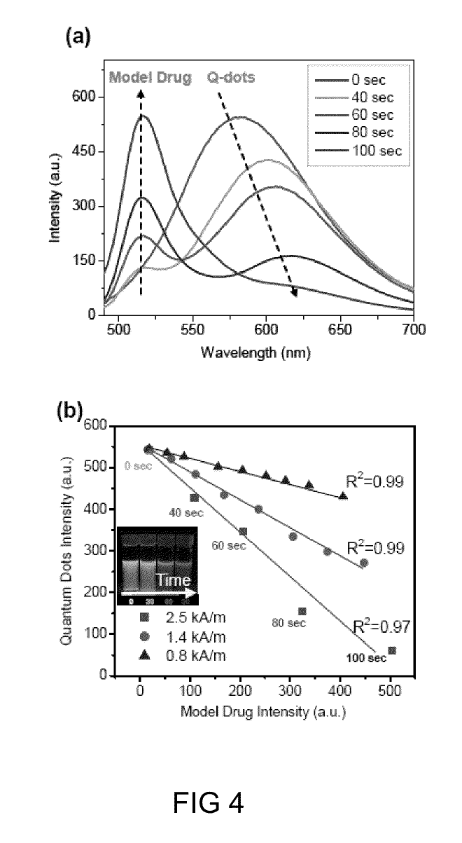

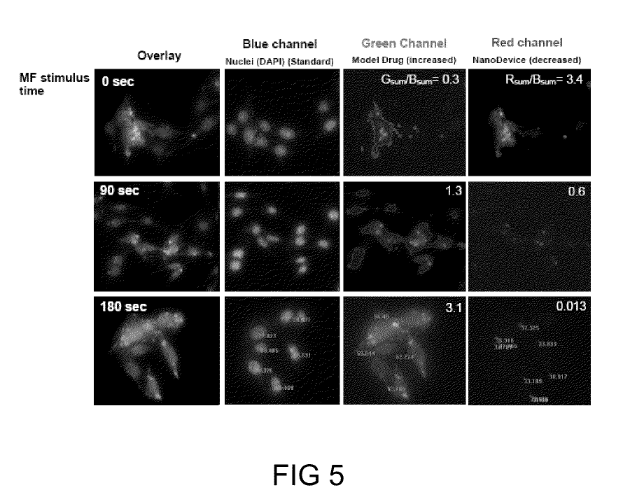

The nanodevices produced by the process described in Example 1 were subjected to a high frequency magnetic field (HFMF) at 50-100 kHz, so as to trigger the release of the encapsulated model drug (i.e., the green fluorescence emitting compound, FITC) from the nanodevices. The HFMF was created by a power supply, a functional generator, an amplifier, and coolant water. Similar equipment was reported in PNAS, vol. 103, 3540-3545 (2006). The strength of the magnetic field depends on the coils. In this example, the coils consist of eight loops. The frequency was set at 50 kHz and the strength of the magnetic field (H) was about 2.5 kA/m. The temperature of the HFMF generator was controlled by cycling water at 25° C. The drug release pattern from 0.05% (Wt %) of magnetic nanodevices was measured in a 20 ml phosphate buffered saline (pH 7.4). PL spectroscopy (PL, Fluorescence Spectrophotometer F-4500, Hitachi, Japan) was used to characterize the release profile of the dye molecule and the fluorescence intensity of nanodevices after application of a high frequency magnetic field (HFMF) of 50 kHz. The nanodevices of Example 1, at a concentration of 0.05% (wt %), were dispersed in the water for varying durations. X-ray photoelectron spectroscopy (XPS) was performed in an ESCALAB 250 (Thermo VG Scientific, West Sussex, UK), equipped with Mg Kα at 1253.6 eV at the anode. The chemical shifts of the XPS peaks were standardized with respect to C 1 s peak at 284.6 eV. Results are illustrated in FIG. 4.

Prior to magnetic stimulation, the FITC-loaded nanospheres showed no sign of release in 24 hours of storage in an ambient environment, which was confirmed via PL spectroscopic monitoring and the observation suggested that the dye may be encapsulated in the core phase for a long period of time without unwanted leakage. However, as illustrated in FIG. 4(a), upon application of HFME for varying lengths of time, the intensity of the green fluorescence emitted from the model drug at 517 nm increased with the duration of the applied magnetic field; whereas the intensity for the QDs decreased with the duration of the applied magnetic field. Effects of varying degrees of HFME on the drug release were also tested on the nanodevices of Example 1, and the results are provided in FIG. 4(b). A linear relationship between the fluorescence intensity and the duration of HFME was found for all 3 doses of HFME that were tested, which indicated that the encapsulated model drug can be released in a controlled manner, that is, by application of a pre-determined dose of HFME for a pre-determined time; and the released model drug can be monitored by the spectra variation of the ZCIS QDs of the nanodevices.

### 1.4 In-Situ Monitoring of Model Drug Release Form the Nanodevices of Example 1

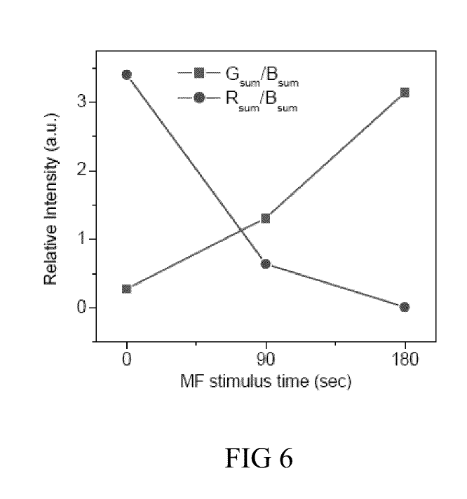

HeLa, human cervical cancer cells, were maintained in Dulbecco's modified Eagle's medium (DMEM) with 10% fetal bovine serum, 100 units/mL penicillin, and 100 μg/mL streptomycin. Cells were cultured at 37° C. in a humidified atmosphere of 5% CO₂ in air. The nanodevices of Example 1 were incubated with the cells for 12 hours. The cells were then subjected to a high frequency magnetic field (HFMF) for 0, 90 and 180 sec. Results were observed by PL microscopy (Nikon TE-2000U, Japan). Digital analysis software (Nikon, Japan) was used to analyze the fluorescence intensities of the model drug and the nanodevices. The conditions of exposure were the same for each color channel. Analysis was done by Nikon C1 software, which defined the fluorescence intensity from 1 to 255. The range of the fluorescence intensities were: Blue channel (60-255), Green channel (40-255), and Red channel (30-255). Results are provided in FIGS. 5 and 6.

As illustrated in FIG. 5, an increase in the duration of the field from 0 sec to 180 sec caused the model drug or the fluorescent compound (Greem channel) to be released rapidly within the cells, while the corresponding fluorescence intensity of ZCIS QDs (Red channel) decreased. Digital software (Nikon, Japan) was used to analyze the fluorescence intensities of both the model drug and ZCIS QDs. Bₛᵤₘ, Gₛᵤₘ and Rₛᵤₘ represent the total intensity of the blue channel, the green channel and the red channel in the images, respectively. The blue fluorescence was due to DAPI dyed nuclei and was expected to be relatively similar for each cell. Therefore, the intensity of the blue channel was used as a standard for each image. Gₛᵤₘ and Rₛᵤₘ are from drug release and quantum dots, respectively. Gₛᵤₘ/Bₛᵤₘ represents the ratio of the green channel intensity to the blue channel intensity, which is an indicative of the relative intensities of the nanodevices in each cell. Result is provided in FIG. 6. The ratios of Gₛᵤₘ/Bₛᵤₘ and Rₛᵤₘ/Bₛᵤₘ versus duration of the magnetic field in the cells gave rise to two separate curves. These curves illustrate that the relative drug concentration, represented by Gₛᵤₘ/Bₛᵤₘ, in the cells increase with the duration of stimulus; whereas the fluorescence intensity of the nanodevices, originated from ZCIS QDs and represented by Rₛᵤₘ/Bₛᵤₘ, decreased in proportion at the same time.

### Example 2

### In Vitro Therapy with Nanodevices Encapsulated Therein Anti-Cancer Agents

### 2.1 Cellular Uptake of Nanodevices of Example 1

Before performing any in vitro therapy, the ability for cells to take up the nanodevices of Example 1 was estimated by confocal microscopy in accordance with steps described below.

Briefly, human lung adenocarcinoma cell line A549 cells were maintained in DMEM (Dulbecco's modified Eagle's medium) containing 10% fetal bovine serum, and 1% penicillin/streptomycin. Cells were cultured with complete medium at 37° C. in a 5% CO₂-humidified atmosphere. A459 cells were plated in 6-well and incubated with FITC-loaded nanodevices of Example 1 for various periods of time, and then washed with phosphate buffered saline (PBS, pH 7.4) for 3 times to remove the excess nanodevices that were not taken up by the cells. Then cells were fixed by 3% formaldehyde and stained with DAPI and Rhodamine-Phalloidin solutions to be observed by confocal microscopy.

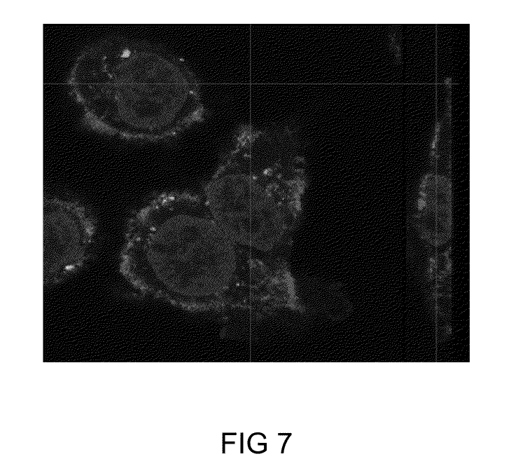

The nanodevices were observed to be localized within the cell in 2 hours (FIG. 7), which substantiates the hypothesis that cells may take up nanodevices fairly easily and efficiently.

### 2.2 In Vitro Cell Toxicity and Cell Viability Effects of Nanodevices

In vitro cytotoxicity of the nanoparticles of Example 1.1 and the nanodevices of Example 1.2 on A549 cells were measured with MTT assay, respectively. Briefly, A549 cells were plated in 96-well plates (10⁴ cells/well), and then exposed to serial concentrations of the nanoparticles of Example 1.1 or the nanodevices of Example 1.2 at 37. At the end of the incubation, 20 ul of MTT solution was added and incubated for another 4 hrs. Then, the medium was replaced with 200 uL of DMSO and the absorbance was monitored using a Sunrise absorbance microplate reader at dual wavelengths of 570 and 650 nm.

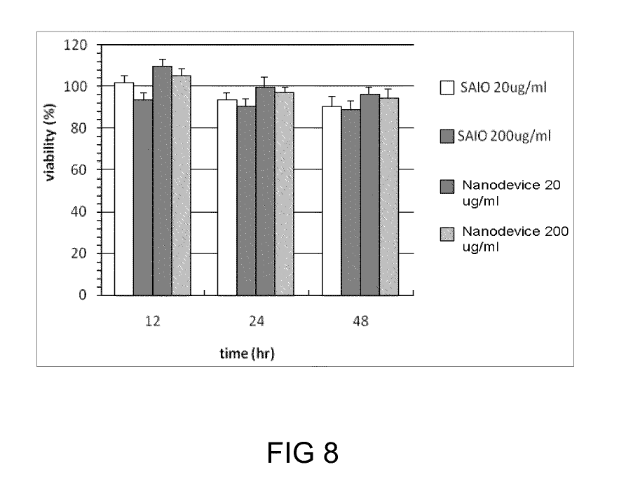

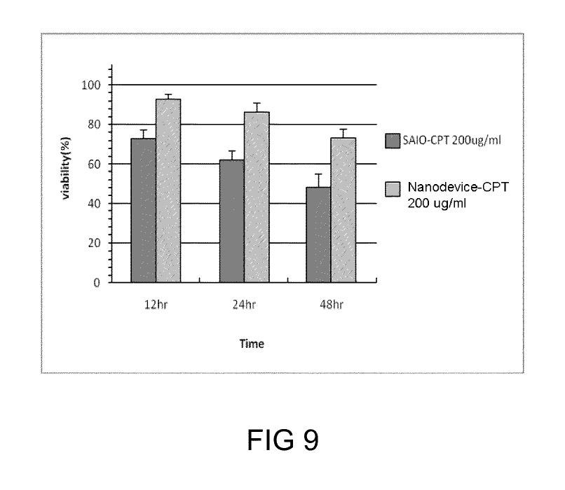

FIG. 8 illustrates the cytotoxicity effects of the nanoparticles of Example 1.1 or the nanodevices of Example 1.2 on A549 cells, and FIG. 8 depicts the viability result. No toxicity was observed for cells incubated with either nanoparticles or nanodevices at a concentration up to 200 μg/ml for 48 hours (FIG. 8). Viability of the cells is about 85% (FIG. 9). The result indicates that the nanoparticles or nanodevices of Example 1 are biocompatible to living cells.

### 2.3 In Vitro Therapy with Nanospheres or Nanodevices Encapsulated Therein Anti-Cancer Drugs

Nanodevices encapsulated with anti-cancer drugs therein were prepared in accordance with the procedures described in Example 1, except camptothein (CPT) was used to replace the green fluorescence compound, FITC. The anti-cancer effect rendered by the CPT-containing nanodevices on cancerous cells was determined by the MTT assay. Briefly, A549 cells were treated with CPT-loaded nanodevices for 6 hours, and then stimulated with various levels of HFMF for various durations, so as to release the encapsulated CPT from the core phase of the nanodevices. Cells were then continued cultured for another 18 hours, and then determined cell viability with MTT assay.

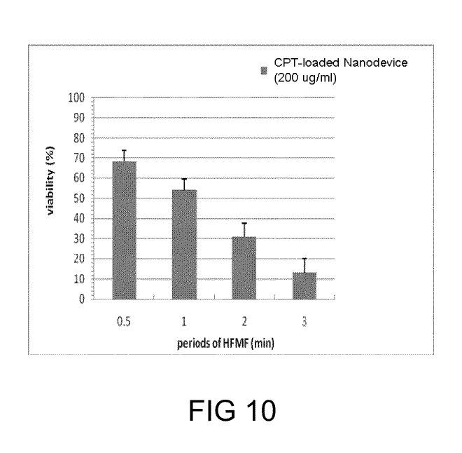

FIG. 10 illustrates the cell viability of A459 cells after being treated with the CPT-loaded nanodevices and magnetically induced to release the encapsulated CPT After cancer cells being treated with CPT-containing nanodevices, cell viability decreased considerably and is believed to be resulting from both heat and drug effects. The heat is believed to be generated from the short-term magnetic treatment for inducing drug release from the nonodevices through interaction with QDs.

The results of this example substantiate the hypothesis that CPT-loaded nanodevices prepared in accordance with the method of this disclosure is an excellent drug delivery system, which displays great biocompatibility, high cell uptake rate, magnetic sensitivity and controlled drug released under HFMF.

### Example 3

### In Vivo Therapy with Nanodevices Encapsulated Therein Anti-Epileptic Agents

### 3.1 Preparation of Ethosuximide (ESM)-Containing Nanodevices

Nanodevices were prepared in accordance with the procedures described in Example 1, except an anti-epileptic agent, ethosuximide (ESM), was used to replace the model drug or the green fluorescence compound, and thereby obtaining ESM-containing nanodevices for in vivo studies.

### 3.2 In-vivo Anti-Epileptic Therapy

Adult male Long-Evans and Wistar rats were used in this study. All rats were kept in a sound-attenuated room under a 12:12 hr light-dark cycle (07:00-19:00 lights on) with food and water provided ad libitum. The experimental procedures were reviewed and approved by the Institutional Animal Care and Use Committee. Briefly, the recording electrodes were implanted under pentobarbital anesthesia (60 mg/kg, i.p.). Subsequently, the rat was placed in a standard stereotaxic apparatus. In total, six stainless steel screws were driven bilaterally into the skull overlying the frontal (A +2.0, L 2.0 with reference to the bregma) and occipital (A −6.0, L 2.0) regions of the cortex to record cortical field potentials. A ground electrode was implanted 2 mm caudal to lambda. Dental cement was applied to fasten the connection socket to the surface of the skull. Following suturing to complete the surgery, animals were given an antibiotic (chlortetracycline) and housed individually in cages for recovery.

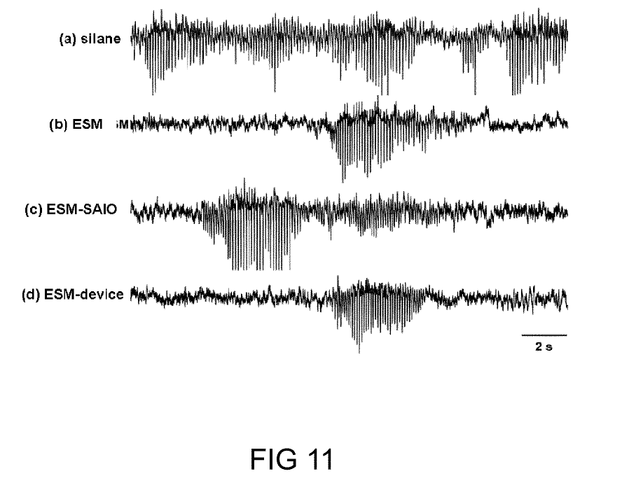

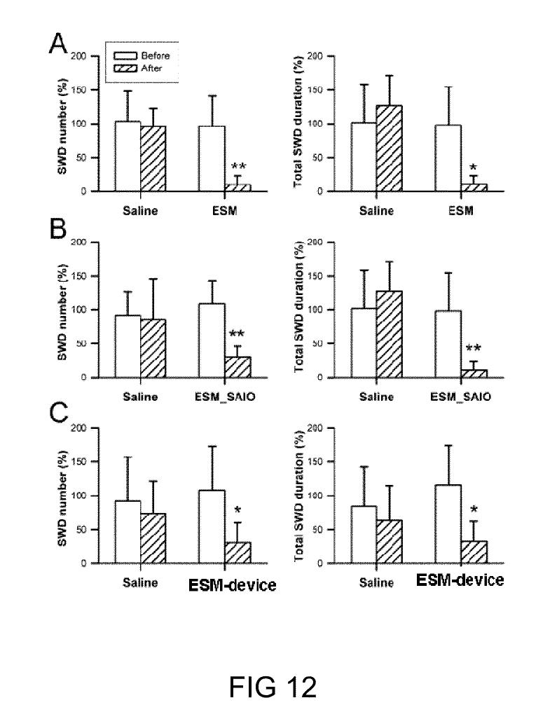

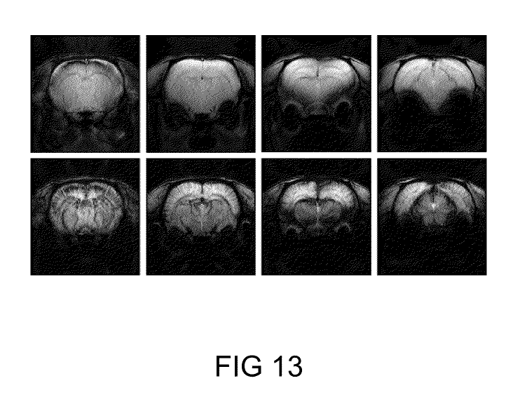

Long-Evans rats are used because they often display spontaneous spike-wave discharges (SWDs), which have been demonstrated to be associated with absence seizures in several aspects of evidence. To confirm the cortical focus theory of SWDs, a pharmacological epileptic rat model, low-dose pentylenetetrazol (PTZ) (20 mg/kg, i.p.) in Wistar rats, was also used. In this preliminary animal test, we compared effect among saline, ethosuximide (ESM), ESM-containing nanoparticles (ESM-SAIO) and ESM-containing nanodevice (ESM-device) in spontaneous SWDs of Long-Evans rats. The chip with dimensions of 5 mm×5 mm×0.02 mm was prepared and implanted into the peritoneum of the rats, while the other three doses were given through IP injection. Results are provided in FIGS. 11 and 12.

FIG. 11 depicts representative examples of spike-wave discharges (SWDs) after the administration of saline, ESM, ESM-SAIO, and ESM-device. The SWDs showed no obvious difference. In this experiment, 1-hour spontaneous brain activity before the treatment (baseline) and another 1-hour spontaneous brain activity 30 minutes after the treatment were recorded, respectively. The indexes were normalized by averaging the two 1-hour baselines. In the conditions of administering ESM-SAIO and ESM-device, rats were restrained in a plastic box then put into the center of a coil followed by magnetic stimulus (2.5 kA/m) to release ESM from the prepared nanospheres or nanodevices. Although it is hard to quantify the amount of the ESM released into the rats, it is surely indicated that the amount of ESM released, from both ESM-SAIO (FIG. 11(c)) and ESM-device (FIG. 11(d)), demonstrated significant effect in reducing the number and total duration of spontaneous SWDs, as comparing to ESM alone (FIG. 11(b)).

SWD number and total SWD duration for animals before and after application of saline, ESM, ESM-SAIO, and ESM-device, respectively are illustrated in FIG. 12. It is clear that different forms of ESM significantly reduced SWD number and total SWD duration.

These in-vivo data, albeit relatively preliminary, evidence that the ESM with the nanoparticle and the chip can be successfully eluted through an external magnetic stimulus, as that observed in vitro. In the meantime, the therapeutic efficacy of the ESM being eluted appeared to suppress SWD.

### Example 4

### In-Vivo Tracking of the Nanodevices by Magnetic Resonance Imaging (MRI)

### 4.1 In-Vitro MRI

Nanodevice samples were prepared in accordance with the steps described in Example 1, except 0.5% PVP was used as the polymeric material, and the final concentration of iron was set at a level of no more than 150 g Fe/mL. For MR imaging, R1 (spin-lattice relaxation rate) and R2 (spin-spin relaxation rate) measurements were performed using 0.47 T nuclear magnetic resonance (NMR). The R1 and R2 of said nanodevice were 63.2 mM⁻¹sec⁻¹ and 372.8 mM⁻¹sec⁻¹ respectively, and both higher than most of the commercial products.

### 4.2 In-Vivo MRI

For in vivo MRI experiments, five adult male Wistar rats of 250-300 g body weight (National Laboratory Animal Center, Taiwan) were used. The animals were initially anesthetized by 3% isoflurane. A PE-50 catheter was inserted in the left femoral vein for subsequent a-chloralose anesthesia (70 mg/kg). Anesthetized rats were positioned on a stereotaxic holder, and body temperature was maintained using a warm-water circulating system. Two ear bars and an incisor fixer were used to position the rat head, with tapes used to restrain the body.

The images were taken by use of the Bruker Biospec BMT 47/40 4.7 T system equipped with an actively shielded gradient system (0-5.9 G/cm in 500 μs). A 20 cm volume coil was used as the RF transmitter and a 2 cm surface coil placed on the head as receiver. A T₂-weighted scout image was scanned in the mid-sagittal plane to localize the anatomical position by identifying the anterior commisure (bregma −0.8 mm). Four slices T₂-weighted template images (bregma −0.8 mm, −2.8 mm, −4.8 mm and −6.8 mm) as shown in FIG. 12 were acquired using spin echo sequence (TR=4000 ms, TE=80 ms, FOV=4 cm, SLTH=2 mm, NEX=2, and the acquisition matrix was 256×128 with a matrix of 256×256 after zero-filling).

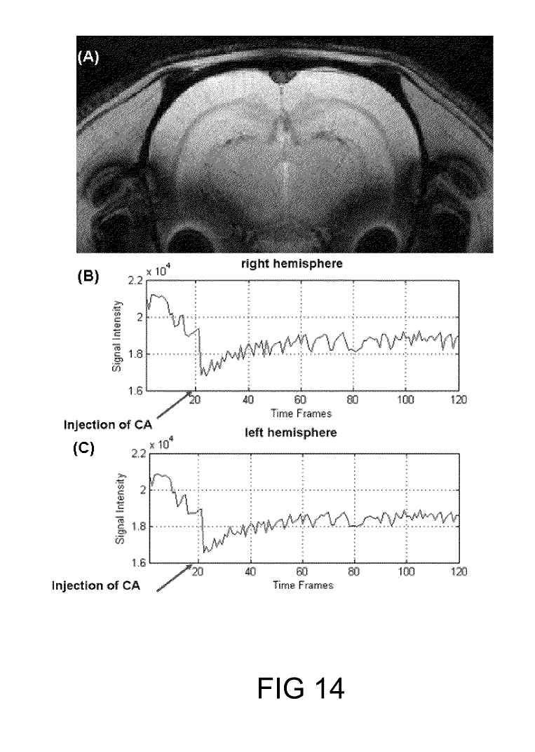

For dynamic perfusion imaging, a 120-repetitive four slices gradient echo images (TR=215 ms, TE=20 ms, flip angle=22.50, FOV=4 cm, SLTH=2 mm, NEX=1 and acquisition matrix was 256×64 with a matrix of 256×256 after zero-filling) were acquired at the same position and each time frame took 13 s. Due to the long blood half life, the nanodevice injection produces stable and long-lasting state for MR scanning. 45% drop in MR signal intensity (FIG. 14) was observed in the brain parenchyma.

### 4.3 In-Vivo Pharmaceutical MRI Diagnosis Using Nanodevices of Example 1 as Contrast Agents

For the pharmaceutical MRI (phMRI) experiment, 40-repetitive gradient echo images (other scanning parameters were identical as above) were acquired and the IOP (30 mg/kg) was injected in the 10th time frame. After 15 min of circulation, 100-repetitive four slices gradient echo images were acquired and the amphetamine (2 mg/kg) was injected in the 20^{th} time frame.

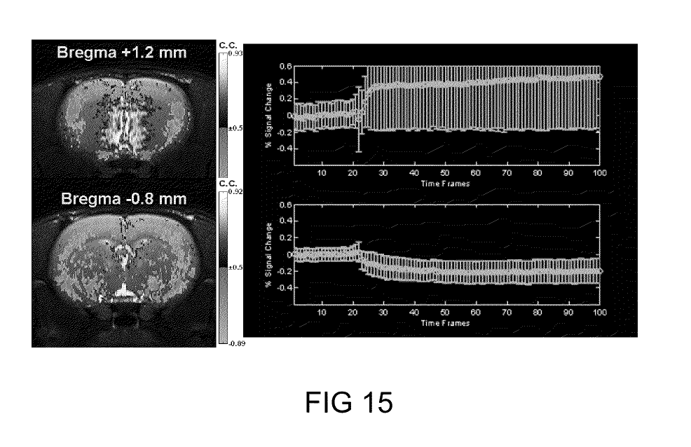

The amphetamine was also used as a functional stimulator to reveal the activated area and the difference of regional cerebral blood volume (rCBV) in alpha-chloralose anesthetized rats. Our study found that the activated areas included striatum, cerebral cortex and thalamus as shown in FIG. 15, and the trend of signal difference were corresponded with the previous dopaminergic stimuli experiments. Furthermore, using nanodevices of Example 1 as a contrast agent revealed the temporal patterns of amphetamine challenge, which provides not only a better contrast-to-noise ratio (CNR), but also allows measurements for meaningful neurovascular responses.

The foregoing description of various embodiments of the invention has been presented for purpose of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of manufacturing a drug delivery nanodevice, comprising:
providing a first solution by dispersing a nanosphere in a first solvent containing therein a zinc salt, wherein the nanosphere and the zinc salt respectively have a concentration of about 1-40 mg/ml and 0.02-0.2 mmol/ml in the first solution;
providing a second solution by mixing at least two quantum dot precursors in a second solvent, wherein each of the at least two quantum dot precursors has a concentration of about 0.003-0.03 mmol/ml in the second solution; and
mixing the first solution with the second solution in the presence of an inert gas at a temperature between 10° C. to 300° C. and thereby forming a quantum dot on the surface of the nanosphere.

2. The method of claim 1, wherein the nanosphere is formed by steps of:
providing a suspension by mixing about 1-10% (wt %) of a polymeric material or an inorganic material with about 0.01-80% (wt %) of a drug in a polar solvent and thereby forming a drug-containing polymeric or inorganic nanoparticle; and
adding at least two metal oxide precursors in a molar ratio of about 2:1 to about 5:1 to the suspension and vigorously stirred for about 2-12 hrs at a temperature of about 20-120° C.;
wherein the at least two metal oxide precursors self-assembly into a metal oxide outer shell around the polymeric or inorganic nanoparticle.

3. The method of claim 2, wherein the polar solvent is water or a C₁₋₆ alcohol.

4. The method of claim 3, wherein the polymeric material is selected from the group consisting of polyvinylpyrrolidone (PVP), polyethylene (PE), polyamide, polyester, polyanhydride, polyether, poly acetal, polysaccharide and phospholipid; and the inorganic material is selected from a group consisting of titania, silica and a compound material made of calcium and phosphate.

5. The method of claim 4, wherein the polysaccharide is any of starch, cellulose, pectin, chitin or chitosan; and the phospholipid is any of phosphatidylcholin (PC), phosphatidylserine (PS), phosphatidylethanolamine, phosphatidylglyerol or phosphatidyllechithin.

6. The method of claim 3, wherein the at least two metal oxide precursors are selected from the group consisting of ferrous chloride (II), ferric chloride (III), cobalt chloride (II), ferrous nitrate (II), ferric acetate (III), cobalt acetate (II), gadolinium chloride (III), and manganous acetate (II).

7. The method of claim 3, wherein the metal oxide outer shell is a singly crystal shell, poly-crystal shell or an amorphous shell that comprises any of Fe₂O₃, Fe₃O₄, CoFe₂O₄, MnFe₂O₄ or Gd₂O₃.

8. The method of claim 6, wherein the at least two metal oxide precursors comprise ferrous chloride (II) and ferric chloride (III), and the metal oxide outer shell is a single-crystal iron oxide shell formed by steps of:
mixing ferrous chloride (II) and ferric chloride (III) in a molar ratio of about 2:1 in water;
adjusting pH to a range between 7 and 12; and
allowing the there formed iron oxide to self-assemble around the polymeric or inorganic nanoparticle.

9. The method of claim 1, wherein the first solvent is a mixture formed by any two solvents selected from the group consisting of trioctylphosphine (TOP), tetrahydrofuran (THF), C₆₋₁₈ alkylene, and dimethylsuloxide (DMSO); and the second solvent is oleylamine or hexadecylamine.

10. The method of claim 1, wherein the zinc salt is diethyldithiocarbamic acid zinc salt.

11. The method of claim 1, wherein the quantum dot is any of CuInZn, CuInS₂, CdS, ZnS or CdTe.

12. The method of claim 11, wherein CuInS₂ is formed from the at least two quantum dot precursors comprising cuprous chloride (I), indium trichloride (III), indium iodide (III), and sulfur powder.

13. The method of claim 11, wherein CuInZn is formed from the at least two quantum dot precursors comprising cuprous chloride (I), indium trichloride (III), indium iodide (III), and zinc stearate.

14. The method of claim 11, wherein CdS is formed from the at least two quantum dot precursors comprising cadmium chloride (II) and sulfur powder.

15. The method of claim 11, wherein CdTe is formed from the at least two quantum dot precursors comprising cadmium chloride (II) and Te powder.

16. The method of claim 11, wherein ZnS is formed from the at least two quantum dot precursors comprising zinc stearate and sulfur powder.

17. The method of claim 1, wherein the temperature is about 140° C.

18. The method of claim 1, wherein the inert gas is any of N₂, He, Ne, Ar or combinations thereof.

19. A drug delivery nanodevice prepared by the method of claim 1.

20. A nanodevice, comprising:
a nanosphere comprising:
a core made of a polymeric material or an inorganic material; and
an outer shell made of a metal oxide; and
a quantum dot deposited on the surface of the outer shell, wherein the quantum dot is selected from the group consisting of CuInZn, CuInS₂, CdS, ZnS and CdTe.

21. The nanodevice of claim 20, wherein the polymeric material is selected from the group consisting of polyvinylpyrrolidone (PVP), polyethylene (PE), polyamide, polyester, polyanhydride, polyether, poly acetal, polysaccharide and phospholipid; and the inorganic material is selected from a group consisting of titania, silica and a compound material made of calcium and phosphate.

22. The nanodevice of claim 20, wherein the metal oxide outer shell is a single crystal shell, poly-crystal shell or an amorphous shell that comprises any of Fe₂O₃, Fe₃O₄, CoFe₂O₄, MnFe₂O₄ or Gd₂O₃.

23. The nanodevice of claim 20, wherein the nanosphere has an average diameter ranges from about 10 nm to 100 nm.

24. The nanodevice of claim 20, wherein the quantum dot is paramagnetic, and the nanodevice is capable of being tracked and imaged by an imaging technique selected from the group consisting of electron spin resonance (ESR) imaging, X-ray imaging, computed tomography and magnetic resonance imaging (MRI).

25. The nanodevice of claim 20, further comprising a drug encapsulated within the core, and the drug is capable of being released from the core upon magnetically stimulating the quantum dot with a magnetic field of about 0.05 kA/m to 2.5 kA/m.

26. The nanodevice of claim 25, wherein the drug is any of an anti-epileptic agent, an anti-tumor agent, an anti-bacterial agent, an anti-viral agent, an anti-proliferative agent, an anti-inflammatory agent, an anti-diabetic agent, or a hormone.

27. The nanodevice of claim 26, wherein the anti-inflammatory agent is any of corticosteroids, ibuprofen, methotrexate, aspirin, salicyclic acid, diphenyhydramine, naproxen, phenylbutazone, indomethacin or ketoprofen.

28. The nanosphere of claim 26, wherein the anti-viral agent is any of acyclovir, ribavirin, zanamivir, oseltamivir, zidovudine or lamivudine.

29. The nanodevice of claim 26, wherein the anti-epileptic agent is any of acetazolamide, carbamazepine, clobazam, clonazepam, diazepam, ethosuximide, ethotoin, felbamate, fosphenytoin, gabapentin, lamotrigine, levetiracetam, mephenytoin, metharbital, methsuximide, methazolamide, oxcarbazepine, phenobarbital, phenytoin, phensuximide, pregabalin, primidone, sodium valproate, stiripentol, tiagabine, topiramate, trimethadione, valproic acid, vigabatrin or zonisamide.

30. The nanodevice of claim 26, wherein the anti-proliferative agent is any of actinomycin, doxorubicin, daunorubicin, valrubicine, idarubicin, epirubicin, bleomycin, plicamycin or mitomycin.

31. The nanodevice of claim 26, wherein the anti-diabetic agent is any of a sulfonylurea, a meglitinide, a biguanide, a thiazolidinedione, an alpha-glucosidase inhibitor, or a peptide analog.

32. The nanodevice of claim 31, wherein the sulfonylureas is any of tolbutamide, acetohexamide, tolazamide, chlorpropamide, glipizide, glyburide, glimepiride or gliclazide; the meglitinide is repaglinide or nateglinide; the biguanide is any of metformin, phenformin, or buformin; the thiazolidinedione is any of rosiglitazone, pioglitazone or troglitazone; the alpha-glucosidase inhibitor is miglitol or acarbose; the peptide analog is any of exenatide, liraglutide, taspoglatide, vildagliptin, sitagliptin or pramlintide.

33. The nanodevice of claim 26, wherein the hormone is any of insulin, epidermal growth factor (EGF), progesterone, estrogen, corticosteroids or androgens.

34. A method for magnetically induced drug release in a subject, comprising:
(a) administering a sufficient amount of the nanodevice of claim 25 to a body portion of the subject; and
(b) magnetically stimulating the body portion of the subject with a magnetic field from about 0.05 kA/m to 2.5 kA/m for a period of about 10 to 180 sec, such that the nanodevice of claim 25 releases the encapsulated agent into the body portion of the subject.

35. The method of claim 34, wherein the subject is a human.

36. The method of claim 34, further comprising a step (c) of tracking the nanodevice in the body portion of the subject by an imaging method selected from the group consisting of ESR imaging, X-ray imaging, computed tomography, and MRI without further addition of a contrast agent.

37. A method for in vivo imaging a subject, comprising:
(a) administering a sufficient amount of the nanodevice of claim 20 to a body portion of the subject; and
(b) tracing the body portion of the subject by an imaging method selected from the group consisting of ESR, X-ray imaging, computed tomography, and MRI without further addition of a contrast agent.
